(19) United States
(12) Patent Application Publication
(10) Pub. No.: US 2007/0282409 A1
(43) Pub. Date: Dec. 6, 2007

(54) Low frequency medical apparatus

(75) Inventors: Shao-Hung Lee, Taipei (TW); Paul Yang, Taipei (TW)

Correspondence Address: BACON & THOMAS, PLLC, 625 SLATERS LANE, FOURTH FLOOR, ALEXANDRIA, VA, 22314, US

(73) Assignee: HEALT & LIFE CO., LTD., CHUNG HO CITY (TW)

(21) Appl. No.: 11/447,106

(22) Filed: Jun. 6, 2006

(51) Int. Cl. A61N 1/00

(52) U.S. Cl. 607/115

(57) ABSTRACT

A low frequency medical apparatus includes a low frequency generator having an electric cord with an end coupled to the low frequency generator for transmitting a pulse current produced by the low frequency to the outside and another end coupled to an electric plug. The electric plug includes an anode and a cathode, and the electric plug is electrically coupled to a conductive fiber area on the fabric. The conductive fiber area is blended with a metal conductor fiber fabric, such that a low frequency pulse current generated by the low frequency generator can be passed through the electric cord to the electric plug and the conductive fiber area on the fabric, and thus the invention can provide a larger patch area than a traditional low frequency generator, so as to increase the area for intermittent contractions and expansions and achieve a better medical treatment effect.

### BACKGROUND OF THE INVENTION

1. Field of the Invention

The present invention relates to a low frequency medical apparatus, and more particularly to a low frequency medical apparatus that connects a low frequency generator with a fabric, and a conductive fiber area on the fabric is blended with a metal conductor and electrically connected to the low frequency generator, such that when the medical apparatus is used, the conductivity of the conductive fiber area on the fabric is larger than that of an area provided by a sticking plate of a prior art low frequency generator, so as to increase the using area and enhance the effect of medical treatments.

2. Description of the Related Art

Since the concept of household nursing and health care gains more attentions in these days, and people take their health more seriously nowadays, health care and medical equipments are introduced constantly to the market. Referring to FIG. 1 for a low frequency medical apparatus, the medical equipment includes a low frequency generator 100, and the low frequency generator 100 has a battery 110 for supplying power, and the battery 110 is coupled to a circuit board 120, and the circuit board 120 is coupled separately to an adjusting button 121 and an electric circuit 122, wherein the adjusting button 121 is used for adjusting the intensity and intermittent time produced by the low frequency, and the electric circuit 122 is coupled separately to anode and cathode patches 130. When the medical apparatus is used, a low frequency current of an intermittent pulse is generated by the low frequency generator 100. With the guide of the anode and cathode patches 130, the skin and acupuncture points in contact with the anode and cathode patches 130 are stimulated to produce intermittent contraction and expansion effects and achieve the massage and medical treatment purposes. Since the stimulating area provided by the patches 130 is very small, the effect of producing contractions and relaxes for the body is relatively small, and such arrangement is ineffective for the medical treatment.

### SUMMARY OF THE INVENTION

In view of the foregoing shortcomings of the prior art apparatuses, the inventor of the present invention based on years of experience in the related field to conduct extensive researches and experiments, and finally developed a low frequency medial apparatus in accordance with the present invention to overcome the shortcomings.

The primary objective of the present invention is to provide a low frequency medial apparatus that blends and weaves a metal conductor with a general textile fiber to produce a fabric, and the fabric is coupled to a low frequency generator, so as to increase the area used for medical treatments.

To achieve the foregoing objective, the present invention provides a low frequency medical apparatus that has a low frequency generator, and the low frequency generator has an electric cord for transmitting a pulse current produced by the low frequency to the outside. Another end of the electric cord is coupled to an electric plug, and the electric plug is comprised of an anode and a cathode electrically coupled to a conductive fiber area on the fabric. The conductive fiber area on the fabric is blended with a metal conductor fiber fabric, such that when the medical apparatus is used, the low frequency pulse current generated by the low frequency generator is passed through the electric cord to the electric plug and the conductive fiber area on the fabric. With the conductivity of the conductive fiber area on the fabric, the invention provides a larger patch area than that provided by the prior art low frequency generator, so as to increase the using area for intermittent contractions and expansions and achieve a better medical treatment effect.

Another objective of the present invention is to provide a low frequency medical apparatus, wherein the fabric has a latch base to facilitate latching the electric plug with the latch base and achieve an electrical connection.

A further objective of the present invention is to provide a low-frequency medical apparatus, wherein the conductive fiber area on the fabric is woven by a plurality of warps and wefts, and the warps or wefts are produced by twisting and enwinding twisted yarns made of general fibers and conductor twisted yarns.

### BRIEF DESCRIPTION OF THE DRAWINGS

The objectives, shape, assembly, structure, characteristic and performance of the present invention will become apparent with the detail description and accompanying drawings as follows:

FIG. 1 is a schematic view of a prior art apparatus;

FIG. 2 is a schematic perspective view of a first preferred embodiment of the present invention;

FIG. 2a is an enlarged view of a fabric of a first preferred embodiment of the present invention;

FIG. 3 is a schematic perspective view of a second preferred embodiment of the present invention;

FIG. 3a is an enlarged view of a fabric of a second preferred embodiment of the present invention;

FIG. 4 is a schematic perspective view of a third preferred embodiment of the present invention;

FIG. 4a is an enlarged view of a fabric of a third preferred embodiment of the present invention;

FIG. 5 is a schematic perspective view of a fourth preferred embodiment of the present invention; and

FIG. 5a is an enlarged view of a fabric of a fourth preferred embodiment of the present invention.

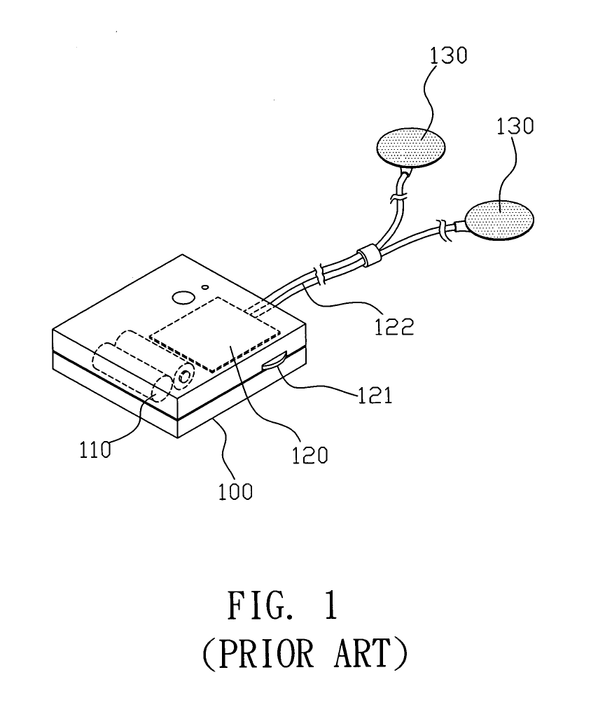

### DETAILED DESCRIPTION OF THE PREFERRED EMBODIMENTS

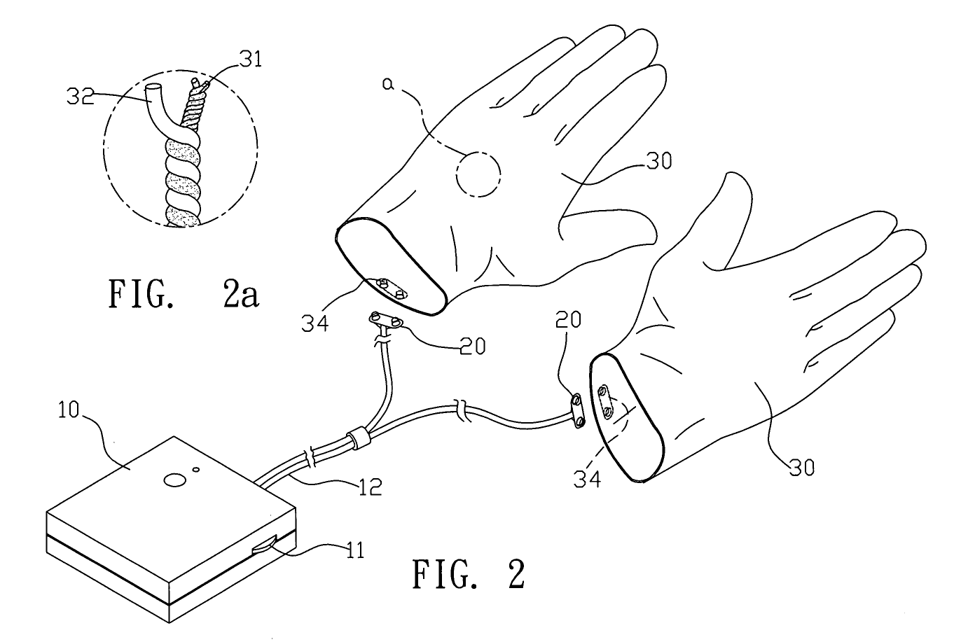

Referring to FIGS. 2 and 2a for a low frequency medical apparatus according to a preferred embodiment of the present invention, the apparatus comprises: a low frequency generator 10; an adjusting button 11 disposed adjacent to the low frequency generator 10 for adjusting the magnitude and intermittent time of a low frequency pulse current produced by the low frequency generator 10; and an electric cord 12 (which includes two power cords as used in this embodiment) coupled to the low -frequency generator 10 for transmitting the pulse current produced by the low frequency generator 10 to the outside, and another end of the electric cord 12 is coupled to an electric plug 20 (which is a latch point as used in this embodiment), and the electric plug 20 is comprised of an anode and a cathode; a fabric 30 (which is a glove as used in this embodiment) being a conductive fiber fabric blended with a metal conductor (as shown in FIG. 2a) and having a latch base 34 latched and electrically coupled to the electric plug 20, and the latch base 34 is electrically coupled to the fabric 30, such that when the medical apparatus is used, the glove is worn on a user's palm, and a low frequency pulse current produced by the low frequency generator 10 is passed through the electric cord 12 to the electric plug 20, latch base 34 and electric circuit 35 and electrically coupled to the fabric 30. With the contractions or expansions of the fabric 30, a patient's pain can be relieved. Further, the medical apparatus of the invention can produce a larger patch area than that of a prior art low frequency generator, so as to increase the area for intermittent contractions and expansions and achieve a better medical treatment effect.

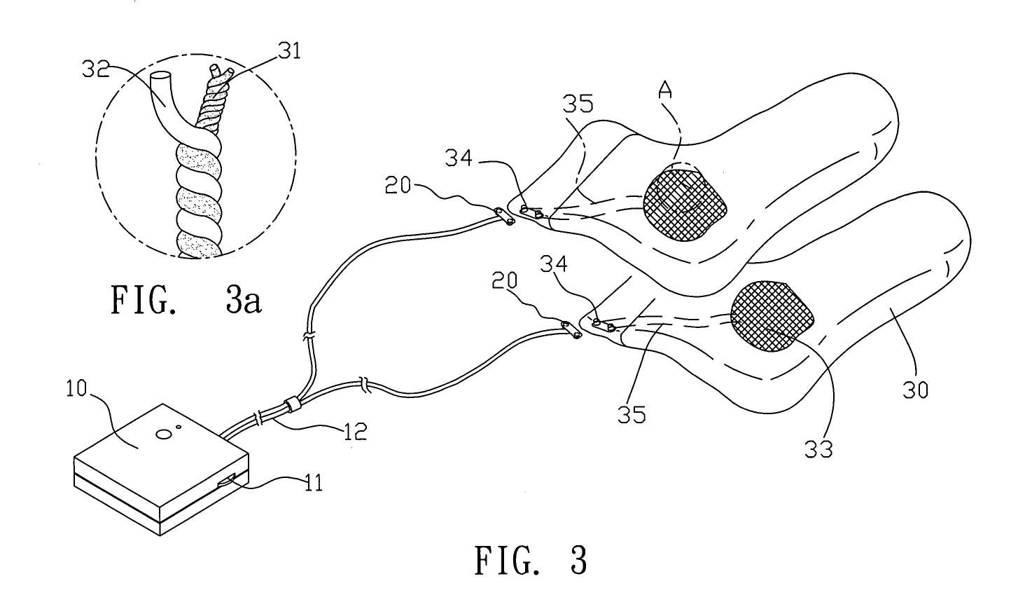

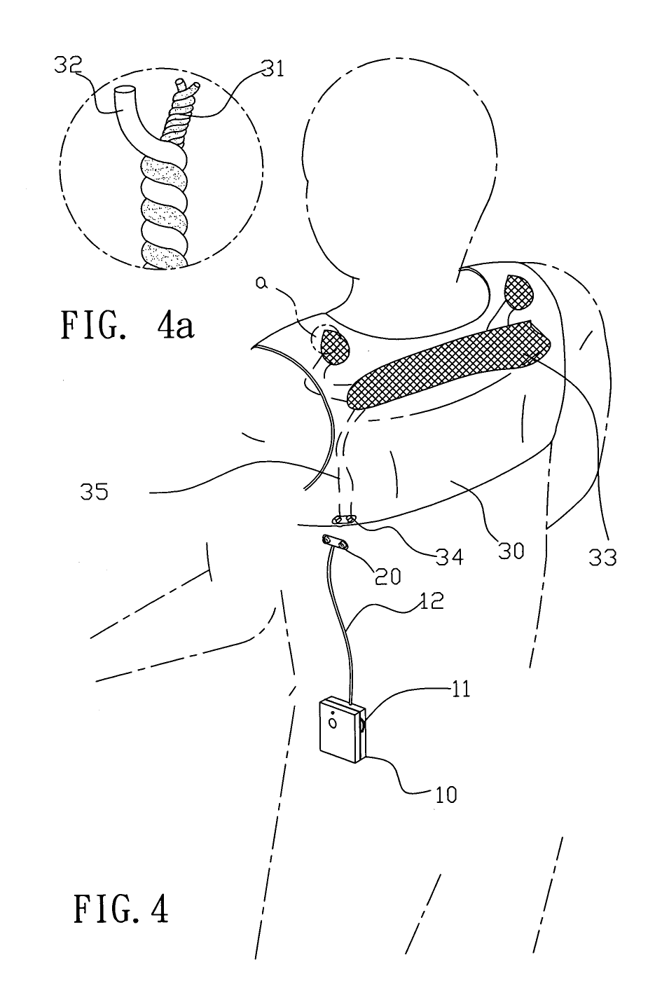

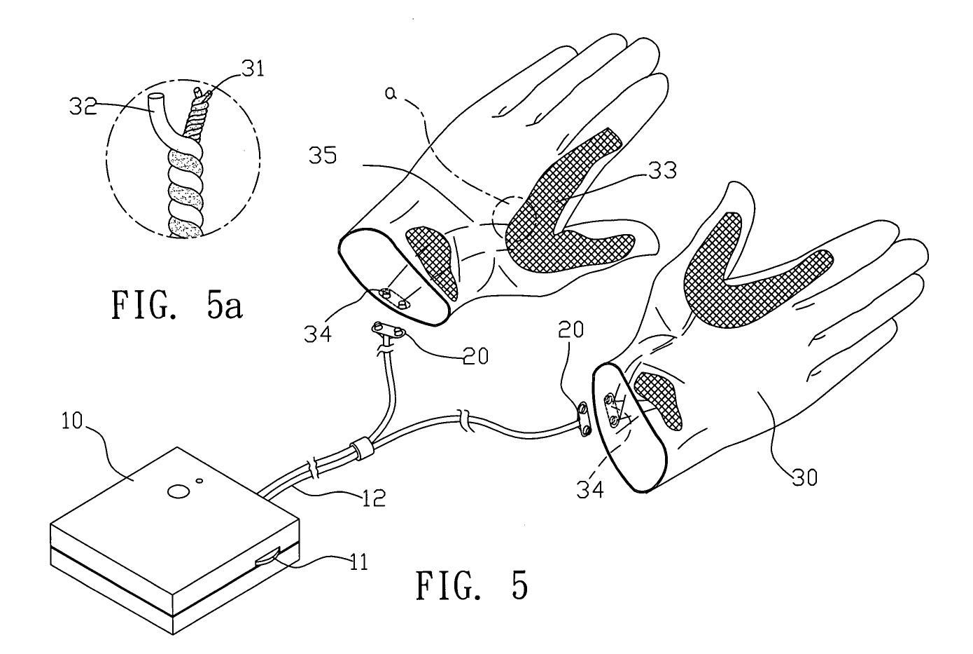

Referring to FIGS. 3, 3a, 4, 4a 5 and 5a for the second, third and fourth embodiments respectively, the medical apparatus comprises: a low frequency generator 10; an adjusting button 11 disposed adjacent to the low frequency generator 10 for adjusting the magnitude and intermittent time of a low frequency pulse current produced by the low frequency generator 10; and an electric cord 12 (which includes two power cords as used in this embodiment) coupled to the low frequency generator 10 for transmitting the pulse current produced by the low frequency generator 10 to the outside, and another end of the electric cord 12 is coupled to an electric plug 20 (which is a latch point as used in this embodiment), and the electric plug 20 is comprised of an anode and a cathode; a fabric 30 (which is a sock, a vest, or a glove as used in the second, third and fourth embodiment respectively) at a portion of the conductive area (such as the acupuncture points of a palm, a sole or a shoulder or any position with pain) being a conductive fiber area 33 blended with a metal conductor (as shown in FIGS. 3a, 4a and 5a). The fabric 30 has a latch base 34 latched and electrically coupled to the electric plug 20, and the latch base 34 is electrically coupled to the conductive fiber area 33 by the metal of the electric circuit 35 in the fabric 30 (wherein the electric circuit is woven in the fabric), such that when the medical is used, a glove, a sock or a vest is selected according to the required massaging position of a palm, a sole or a shoulder respectively and worn at the conductive fiber area 33 corresponding to a desired position. Thus, a low frequency pulse current produced by the low frequency generator 10 is passed through the electric cord 12 to the electric plug 20, the latch base 34 and the electric circuit 35 and electrically coupled to the conductive fiber area 33. With the contractions or expansions of the conductive fiber area 33, a patient's pain can be relieved, and thus the invention can provide a larger patch area than a traditional low frequency generator, so as to increase the area for intermittent contractions and expansions and achieve a better medical treatment effect.

Referring to FIGS. 2a, 3a, 4a and 5a, the conductive fiber area 33 on the fabric is woven by a plurality of warps and wefts, and the warps or wefts are produced by twisting and enwinding twisted yarns 31 made of general fibers and metal conductor twisted yarns 32 (as shown in FIGS. 2a, 3a, 4a and 5a), and the metal conductor twisted yarn 32 could be a copper wire or an iron wire, etc.

In summation of description above, the article, shape, assembly and structure of the present invention are novel and provides a feasible solution to overcome the shortcomings of the prior art, and a patent application is duly filed accordingly.

However, it is to be noted that the preferred embodiments disclosed in the specification and the accompanying drawings are not intended to limit the invention. To the contrary, it is intended to cover various modifications and similar arrangements and procedures, and thus the scope of the appended claims should be accorded the broadest interpretation so as to encompass all such modifications and similar arrangements and procedures.

What is claimed is:

1. A low frequency medical apparatus, comprising:
a low frequency generator;
an electric cord, coupled to said low frequency generator for transmitting a pulse current produced by said low frequency to the outside;
an electric plug, electrically coupled to said electric cord; and
a fabric, blended with an electric conducting metal fabric, and including a latch base disposed on said fabric electrically coupled to said electric plug, and said latch base being coupled to an electric conducting metal in said fabric, such that said fabric is electrically coupled to said low frequency generator for increasing the area for medical treatments;
thereby, the conductivity of said fabric and an electric connection of said low frequency pulse current produced by said low frequency generator produce intermittent contractions and expansions for said fabric to increase the using area and enhance the effect of medical treatments.

2. The low frequency medical apparatus of claim 1, wherein said fabric is woven by a plurality of warps and wefts, and said warps or wefts are produced by twisting and enwinding twisted yarns made of general fibers and conductor twisted yarns.

3. A low frequency medical apparatus, comprising:
a low frequency generator;
an electric cord, coupled to said low frequency generator, for transmitting a pulse current produced by a low frequency to the outside;
an electric plug, electrically coupled to said electric cord;
a fabric, including a latch base disposed on said fabric and electrically coupled to said electric plug, and said latch base being coupled to a metal electric circuit woven in said fabric;
a conductive fiber area, disposed on a specific area of said fabric and mixed with an electric conducting metal, and electrically coupled to a low frequency pulse current produced by said low frequency generator by said metal electric circuit woven in said fabric and said latch base, such that intermittent contractions and expansions are produced in said conductive fiber area for increasing the using area and enhancing the medical treatment effect.

4. The low frequency medical apparatus of claim 3, wherein said conductive fiber area is made by weaving a plurality of warps and wefts, and said warps or wefts are produced by twisting and enwinding twisted yarns made of general fibers and conductor twisted yarns.

5. The low frequency medical apparatus of claim 3, wherein said conductive fiber area corresponds to a related position of a human body.

6. The low frequency medical apparatus of claims 1 or 3, wherein said fabric is a shoulder strap, a glove or a sock.
